(19) United States
(12) Patent Application Publication
(10) Pub. No.: US 2004/0073081 A1
(43) Pub. Date: Apr. 15, 2004

(54) Probe for dielectric and optical diagnosis

(76) Inventor: Werner Schramm, Fredersdorf (DE)

Correspondence Address: Law Offices of Karl Hormann, PO Box 381516, Cambridge, MA, 02238, US

(21) Appl. No.: 10/469,337

(22) PCT Filed: Feb. 27, 2001
(86) PCT No.: PCT/EP01/02194

(51) Int. Cl. A61B 1/00

(52) U.S. Cl. 600/101

(57) ABSTRACT

An endoscopic measuring probe for medical diagnosis providing an array by means of which both dielectric and optical parameters can be detected at a given site of the tissue during measurement. The measuring probe is formed by a coaxial array of metal outer sheaths and a metal inner conductor, wherein the dielectric consists of light conducting fibers.

The invention relates to an endoscopic measuring probe for medical diagnostics for simultaneously detecting dielectric and optical parameters at the same measuring site. The field of application relates to the diagnosis of tumors measuring the surface of the tissue as well as measuring by penetration into the surface.

Several measuring systems have become known for executing multiple measurements of tissue to distinguish tumorous tissue from healthy tissue.

For instance, U.S. Pat. No. 5,800,350 describes a measuring probe which by contacting the tissue makes it possible to detect a plurality of different physical parameters of the tissue, including electrical and optical ones.

The electrodes for electrical measurements positioned at the front surface of the measuring probe are structured and arranged such that upon placing the probe on the tissue, different resistance paths between a center electrode and separate electrode segments at the periphery are measured to be compared to ensure a reproducible position of the probe on the tissue. Decay rates of current pulses sent through the tissue may also be evaluated.

For detecting the optical properties of the tissue, for which purpose wavelengths of 540, 650, 660, 940 and 1,300 nm are considered to be of particularly high diagnostic value, light conducting fibers are provided in the probe which carry light from light sources to the tissue and return it to corresponding receivers.

German patent specification DE 198 54 292 proposes a multiple parameter measuring system in which the measuring probe used is provided with a separate coaxial cable and, laterally offset therefrom, various light conducting fibers for optical spectroscopy.

The disadvantage of the two mentioned measuring systems is that the measuring site for the impedance measurement does not precisely coincide with the site for the optico-spectroscopic measurements.

It is thus an object of the invention to provide a system which during performance of a measurement makes possible simultaneous detection of dielectric as well as optical parameters at a predetermined site.

The object is accomplished by an arrangement in accordance with claim 1. Advantageous embodiments are the subject of the sub-claims.

By the combination in accordance with the invention of electric wave conductors and optical light conducting fibers in the measuring probe, several diagnostic parameters may be accurately detected at the same tissue area. This is of decisive importance for accurately distinguishing between a tumor and healthy tissue. The small required space advantageously facilitates the fabrication of very thin endoscopic measuring probes.

The invention will be explained in greater detail on the basis of embodiments.

In the drawings:

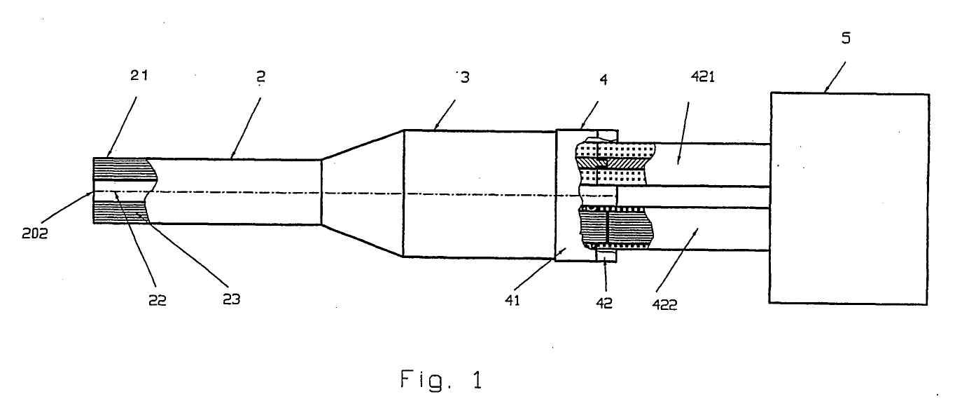

FIG. 1 depicts the principle of a measuring system in which the endoscopic measuring probe is connected to an operating apparatus by a coupling module;

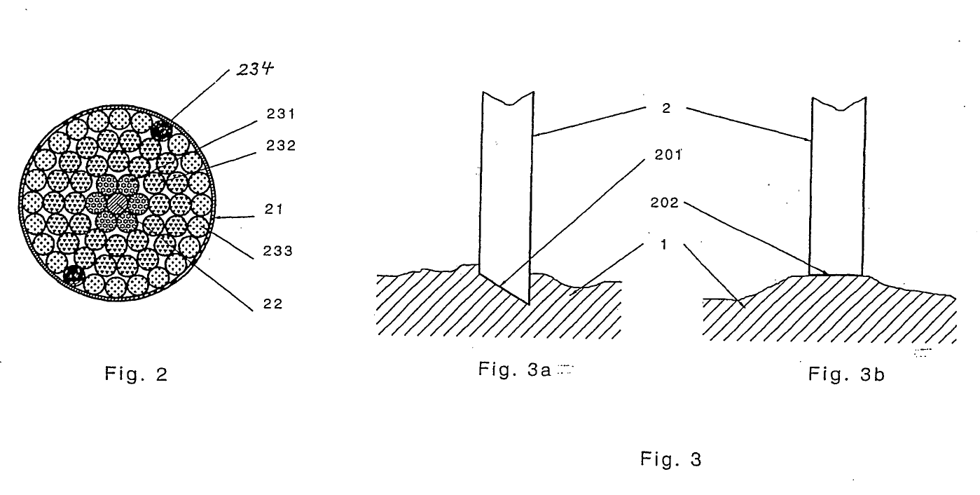

FIG. 2 shows the distribution of light conducting fibers of different function over the cross-section of a coaxial arrangement;

FIG. 3 depicts examples of applications for contact measurements of a tissue

3a invasively; and

3b non-invasively, engagingly.

FIG. 1 depicts the principle structure of a measuring system for the spatially precise dielectric and optico-spectroscopic diagnosis of biological fiber. In addition to the cylindrical endoscopic measuring probe 2 including shaft 3 and coupling module 4, it shows the operating apparatus 5 connected by a coaxial cable 421 and a light conducting fiber 422.

The measuring probe is structured as a combination electrical wave conductor and light conducting fiber probe. The space between the metallic exterior sheath 21 and the coaxial internal conductor 22 is filled by a dielectric substance of light conducting fibers 23. The wave resistance z of the system should be selected such that it corresponds to the wave resistance of the coaxial cable connected by the coupling module 4.

As is well known (Meinke, Grundiach: Handbuch der Hochfrequenztechnik, 3rd Edition, Springer, 1968, page 255), the wave resistance z is calculated on the basis of the diameter D of the exterior sheath 21, the internal diameter d of the internal conductor 22 and the dielectric constant ε_{T} by the equation

z=60/ε_{T}^{-1/2} In D/d.

At its distal end, the measuring probe 2 terminates in a shaft 3 to which is connected a coupling module 4 which consists of a connector 41 with a female connector portion 42. The coupling module 4 in turn is connected by a coaxial cable 421 and light conducting cable 422 to an operating apparatus 5 required for the operation of the measuring probe. The operating apparatus contains signal sources and signal receivers for dielectric and optical spectroscopy.

FIG. 2 depicts a cross-section of the measuring probe in accordance with the invention, which is provided with differently functioning light conducting fibers. The light conducting fibers are uniformly distributed over the cross-section to ensure the characteristics of a symmetrical wave conductor. 231 represents excitation fibers for carrying excitation light of, for instance, 337 nm to the tissue; 232 are fibers for measuring the fluorescence of the tissue; 233 are fibers which detect the scattering of excitation light in the tissue; and 234 depicts fibers for surface Raman spectroscopy.

FIG. 3 depicts two cases of application of differently structured front surfaces of the measuring probe 2 in accordance with the invention. FIG. 4a shows an endoscopic measuring probe 2 having a front surface 201 structured as a point for use in invasive measurements within the tissue 1. FIG. 4b depicts the case of application in which the measuring probe 2 has a planar front surface 202 to be placed on the tissue 1 for performing measurements.

### List of Reference Characters

1 tissue

2 measuring probe

201 front surface structured as a point

202 planar front surface

21 metallic exterior sheath

22 metallic internal conductor

23 light conducting fibers

231 excitation fibers

232 fibers for measuring fluorescence

233 fibers for measuring remission

234 fibers for surface Raman spectroscopy

3 shaft of the endoscopic measuring probe

4 coupling module

41 plug portion

42 female connector portion

421 coaxial cable

422 light conducting fiber

5 apparatus for operating the measuring probe

1. An endoscopic measuring probe for multi parameter diagnostics of biological tissue, characterized by the fact that a cylindrical measuring head (2) structured as a coaxial wave conductor for contacting the tissue (1) to be examined, is provided with a metallic external sheath (21) and a coaxial internal conductor (22), the space between the two being filled by a dielectric substance of light conducting fibers (23) which as groups or individually transmit light of predetermined wavelength from signal sources to the tissue or away from them to optical receivers, the measuring head at its distal end terminating in a shaft (3) with a coupling module (4) which, consisting of a connector (41) with a female connector portion (42), constitutes a separable interface to the operating apparatus (5).

2. The endoscopic measuring probe of claim 1, characterized by the fact that the light conducting fibers as a dielectric substance are uniformly distributed over the cross-section.

3. The endoscopic measuring probe of claim 1 and 2, characterized by the fact that its proximal end is structured as a point (201) for penetration into the tissue (1).

4. The endoscopic measuring probe of claim 1 and 2, characterized by the fact that its proximal end is provided with a planar front surface (202).
